(19) United States
(12) Patent Application Publication
(10) Pub. No.: US 2013/0317596 A1
(43) Pub. Date: Nov. 28, 2013

(54) BIORESORBABLE POLYMER SCAFFOLD AND TREATMENT OF CORONARY ARTERY LESIONS

(71) Applicant: Abbott Cardiovascular Systems Inc., Santa Clara, CA (US)

(72) Inventors: Richard RAPOZA, San Francisco, CA (US); Susan VELDHOF, Rotterdam (NL); Yunbing WANG, Sunnyvale, CA (US)

(21) Appl. No.: 13/842,609

(22) Filed: Mar. 15, 2013

Related U.S. Application Data
(60) Provisional application No. 61/775,424, filed on Mar. 8, 2013.
(60) Provisional application No. 61/768,394, filed on Feb. 22, 2013.
(60) Provisional application No. 61/615,185, filed on Mar. 23, 2012.

(51) Int. Cl. A61F 2/86

(52) U.S. Cl. CPC A61F 2/86
USPC 623/1.16

(57) ABSTRACT

Methods of treating coronary heart disease with bioresorbable polymer stents are described.

This application claims the benefit of U.S. Patent Application No. 61/615,185 filed Mar. 23, 2012, U.S. Patent Application No. 61/768,394 filed Feb. 22, 2013, and U.S. Patent Application No. 61/775,424 filed Mar. 8, 2013, all of which are incorporated by reference herein.

### BACKGROUND OF THE INVENTION

1. Field of the Invention

This invention relates to bioresorbable polymer scaffolds and methods of treatment of coronary lesions with bioresorbable polymer scaffolds

2. Description of the State of the Art

This invention relates generally to methods of treatment with radially expandable endoprostheses, that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffold gets its name because it physically holds open and, if desired, expands the wall of a passageway in a patient. Typically, stents are capable of being compressed or crimped onto a catheter so that they can be delivered to and deployed at a treatment site.

Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty.

Stents are used not only for mechanical intervention but also as vehicles for providing biological therapy. Biological therapy uses medicated stents to locally administer a therapeutic substance. The therapeutic substance can also mitigate an adverse biological response to the presence of the stent. A medicated stent may be fabricated by coating the surface of either a metallic or polymeric scaffolding with a polymeric carrier that includes an active or bioactive agent or drug. Polymeric scaffolding may also serve as a carrier of an active agent or drug.

The stent must be able to satisfy a number of mechanical requirements. The stent must have sufficient radial strength so that it is capable of withstanding the structural loads, namely radial compressive forces imposed on the stent as it supports the walls of a vessel. Radial strength, which is the ability of a stent to resist radial compressive forces, relates to a stent's radial yield strength and radial stiffness around a circumferential direction of the stent. A stent's “radial yield strength” or “radial strength” (for purposes of this application) may be understood as the compressive loading, which if exceeded, creates a yield stress condition resulting in the stent diameter not returning to its unloaded diameter, i.e., there is irrecoverable deformation of the stent. When the radial yield strength is exceeded the stent is expected to yield more severely and only a minimal force is required to cause major deformation.

Once expanded, the stent must adequately provide lumen support during a time required for treatment in spite of the various forces that may come to bear on it, including the cyclic loading induced by the beating heart. In addition, the stent must possess sufficient flexibility with a certain resistance to fracture.

Stents made from biostable or non-degradable materials, such as metals that do not corrode or have minimal corrosion during a patient's lifetime, have become the standard of care for percutaneous coronary intervention (PCI) as well as in peripheral applications, such as the superficial femoral artery (SFA). Such stents have been shown to be capable of preventing early and later recoil and restenosis.

In order to effect healing of a diseased blood vessel, the presence of the stent is necessary only for a limited period of time, as the artery undergoes physiological remodeling over time after deployment. The development of a bioabsorbable stent or scaffold could obviate the permanent metal implant in vessel, allow late expansive luminal and vessel remodeling, and leave only healed native vessel tissue after the full resorption of the scaffold. Stents fabricated from bioresorbable, biodegradable, bioabsorbable, and/or bioerodable materials such as bioabsorbable polymers can be designed to completely absorb only after or some time after the clinical need for them has ended. Consequently, a fully bioabsorbable stent can reduce or eliminate the risk of potential long-term complications and of late thrombosis, facilitate non-invasive diagnostic MRI/CT imaging, allow restoration of normal vasomotion, provide the potential for plaque regression.

### SUMMARY OF THE INVENTION

Embodiments of the invention include a method of treating vascular disease in a patient comprising: deploying a bioabsorbable polymer scaffold composed of a plurality of struts at a segment of an artery of a patient, wherein the segment comprises a scaffolded segment between a proximal and a distal end of the scaffold, a proximal segment proximally adjacent to the proximal end of the scaffold, and a distal segment distally adjacent to the distal end of the scaffold, wherein the proximal segment exhibits constrictive remodeling between baseline and two years after the deployment, wherein the constrictive remodeling comprises a decrease in a cross-sectional area of the proximal segment.

Embodiments of the invention include a method of treating vascular disease in a patient comprising: deploying a bioabsorbable polymer scaffold composed of a plurality of struts at a segment of an artery of a patient, wherein the segment comprises a scaffolded segment between a proximal and a distal end of the scaffold, a proximal segment proximally adjacent to the proximal end of the scaffold, and a distal segment distally adjacent to the distal end of the scaffold, wherein a content of fibrotic and fibrofatty (FF) tissue increases at the distal segment between baseline and two years after the deployment.

Embodiments of the invention include a method of treating vascular disease in a patient comprising: deploying a bioabsorbable polymer scaffold composed of a plurality of struts at a segment of an artery of a patient, wherein the segment comprises a scaffolded segment between a proximal and a distal end of the scaffold, a proximal segment proximally adjacent to the proximal end of the scaffold, and a distal segment distally adjacent to the distal end of the scaffold, and wherein at baseline there is a difference in a compliance of the scaffolded segment between the proximal segment and the distal segment.

Embodiments of the invention include a method of treating vascular disease in a patient comprising: deploying a bioabsorbable polymer scaffold composed of a plurality of struts at a segment of an artery of a patient, the polymer scaffold expanding during deployment which expands the segment to a target diameter, wherein vasomotion of the segment of the artery reappears after deployment due to the replacement of the polymer by de novo formation of malleable tissue comprising proteoglycan, wherein two years after deployment the scaffold area or volume has decreased by less than 10%.

Embodiments of the invention include a method of treating vascular disease in a patient comprising: deploying a bioabsorbable polymer scaffold composed of a plurality of struts at a segment of an artery of a patient, the polymer scaffold expanding during deployment which expands the segment to a target diameter, wherein a neointimal area increases and a mean scaffold area increase between baseline and 1 year and between one year and three years after baseline.

Embodiments of the invention include a method of treating vascular disease in a patient comprising: deploying a bioabsorbable polymer scaffold composed of a plurality of struts at a segment of an artery of a patient, the polymer scaffold expanding during deployment which expands the segment to a target, wherein a total plaque area in the segment increases between baseline and one year and then decreases between one and three years after deployment.

Embodiments of the invention include a method of treating vascular disease in a patient comprising: deploying a bioabsorbable polymer scaffold composed of a plurality of struts at a segment of an artery of a patient, the polymer scaffold expanding during deployment which expands the segment to a target diameter, and wherein a dense calcium percent and a hyperechogenic area of the segment decreases between baseline and 1 year and between one year and three years.

Embodiments of the invention include a method of treating vascular disease in a patient comprising: deploying a bioabsorbable polymer scaffold composed of a plurality of struts at a segment of an artery of a patient, the polymer scaffold expanding during deployment which expands the segment to a target diameter, wherein 3 years after deployment, the segment comprises: return of vasomotion to the segment; enlargement of the scaffold area and mean lumen area between baseline and three years; an increase of neointima in the segment between baseline and three years; and a reduction of plaque area between baseline and three years.

### INCORPORATION BY REFERENCE

All publications patents, and patent applications mentioned in this specification are herein incorporated by reference to the same extent as if each individual publication, patents, or patent application was specifically and individually indicated to be incorporated by reference, and as if each said individual publication, patents or patent application was fully set forth, including any figures, herein.

### BRIEF DESCRIPTION OF THE DRAWINGS

FIG. 1 depicts an exemplary stent scaffold.

FIGS. 2A-B depict the Abbott Vascular Inc. BVS revision 1.1 scaffold.

FIG. 3 depicts an exemplary stent pattern shown in a planar or flattened view.

FIG. 4 depicts a schematic view of a scaffold deployed in a vessel segment showing the scaffolded segment, proximal edge segment, and distal edge segment.

FIG. 5 depicts IVUS-VH images of a distal edge segment, scaffolded segment, and a proximal edge segment at baseline and 1 year follow-up.

FIG. 6 depicts the change in the vessel, lumen, and plaque cross sectional area along a distal edge and a proximal edge of an implanted scaffold at 1 year follow-up.

FIG. 7 depicts the tissue composition along the distal edge and the proximal edge at 1 year follow-up of an implanted scaffold.

FIG. 8 shows a schematic of a cross section of a scaffold deployed in a vessel showing a scaffolded segment, a proximal segment, and a distal segment.

FIG. 9 depicts the mean of the maximum strain values for each of a scaffolded segment, a proximal segment, and a distal segment.

FIG. 10 depicts the compliance in each of the segments of FIG. 9 pre-implantation, post-implantation, and 1 year follow-up.

FIG. 11 shows that the percent of struts uncovered by an endothelial layer decreases between 1 and 3 years from baseline.

FIG. 12 depicts the neointimal area, mean scaffold area, and mean lumen area from OCT for 19 patients between 1 and 3 years.

FIG. 13 shows the serial quantitative IVUS analysis of the total plaque area (uppermost curve), mean scaffold area (middle curve), and mean lumen area (lowermost curve) for Group B2 between baseline and 3 years after baseline.

FIG. 14A depicts IVUS-GS and Echogenicity images for Group B2 at baseline, 1 year, and 3 years.

FIG. 14B depicts the change in percentage hyper-echogenic area (HEA) for ABSORB 1.1, Cohorts B1 (uppermost curve) and B2 (middle curve), and ABSORB 1.0 Cohort A (bottom curve) between baseline, 6 months, 1 year, 2 years, and 3 years.

FIG. 15 shows the evolution of late luminal loss over time for ABSORB Cohort B at 1 year versus ABSORB at 3 year follow-up for Cohort B of 56 patients.

FIG. 16 shows the evolution of late luminal loss over time for ABSORB Cohort B at 1 year (lighter color dots) versus ABSORB at 3 years (darker color dots).

FIG. 17 shows the evolution of late luminal loss over time for ABSORB Cohort B at 3 years (darker color dots) follow-up versus Xience at 2 years follow-up (lighter color dots) everolimus eluting stent.

FIG. 18 shows the mean lumen diameter before and after addition of nitrate, a vasodilator, sometime after baseline in the scaffolded segment.

FIG. 19A-D depicts QCA results for the evolution of late luminal loss over time for ABSORB Cohort B at 6 months, 1 year, 2 years, and 3 years follow-up.

FIG. 20 is table of results of quantitative IVUS analysis for ABSORB Cohort B for 6 months, 1 year, 2 years, and 3 years follow-up.

FIG. 21 depicts serial quantitative IVUS analysis of the mean vessel area, mean scaffold area, mean lumen area, and mean plaque area for Group B1 between baseline and 2 years and Group B2 between baseline and 3 years follow-up.

FIG. 22 depicts the results of serial IVUS-VH analysis for percent of dense calcium for Group B1 between baseline and 2 years and Group B2 between baseline and 36 months follow-up.

FIG. 23 depicts changes in percentage hyper-echogenic area (HEA) for ABSORB 1.1, Cohorts B1 and B2 between post-procedure and 3 year follow-up.

FIG. 24 is a table including results for ABSORB Cohort B of quantitative OCT analysis post-procedure and for 1 year and 3 years follow-up.

FIG. 25 is a table including results for ABSORB Cohort B for mean scaffold area, mean lumen area, and mean neointimal area from quantitative OCT analysis for 6 months, 1 year, 2 years, and 3 years follow-up.

### DETAILED DESCRIPTION OF THE INVENTION

Various embodiments of the present invention include treatment of coronary artery disease with bioresorbable polymer stents. The bioresorbable stents can include a support structure in the form of a scaffold made of a material that is bioresorbable, for example, a bioresorbable polymer such as a lactide-based polymer. The scaffold is designed to completely erode away from an implant site after treatment of an artery is completed. The scaffold can further include a drug, such as an antiproliferative or anti-inflammatory agents. A polymer coating disposed over the scaffold can include the drug which is released from the coating after implantation of the stent. The polymer of the coating is also bioresorbable.

The present invention is applicable to, but is not limited to, self-expandable stents, balloon-expandable stents, stent-grafts, and generally tubular medical devices in the treatment of artery disease. The present invention is further applicable to various stent designs including wire structures, and woven mesh structures.

Self expandable or self expanding stents include a bioabsorbable polymer scaffold that expands to the target diameter upon removal of an external constraint. The self expanding scaffold returns to a baseline configuration (diameter) when an external constraint is removed. This external constraint could be applied with a sheath that is oriented over a compressed scaffold. The sheath is applied to the scaffold after the scaffold has been compressed by a crimping process. After the stent is positioned at the implant site, the sheath may be retracted by a mechanism that is available at the end of the catheter system and is operable by the physician. The self expanding bioabsorbable scaffold property is achieved by imposing only elastic deformation to the scaffold during the manufacturing step that compresses the scaffold into the sheath.

The bioabsorbable scaffold may also be expanded by a balloon. In this embodiment the scaffold is plastically deformed during the manufacturing process to tightly compress the scaffold onto a balloon on a catheter system. The scaffold is deployed at the treatment site by inflation of the balloon. The balloon will induce areas of plastic stress in the bioabsorbable material to cause the scaffold to achieve and maintain the appropriate diameter on deployment.

A stent scaffold can include a plurality of cylindrical rings connected or coupled with linking elements. For example, the rings may have an undulating sinusoidal structure. When deployed in a section of a vessel, the cylindrical rings are load bearing and support the vessel wall at an expanded diameter or a diameter range due to cyclical forces in the vessel. Load bearing refers to the supporting of the load imposed by radial inwardly directed forces. Structural elements, such as the linking elements or struts, are generally non-load bearing, serving to maintain connectivity between the rings. For example, a stent may include a scaffold composed of a pattern or network of interconnecting structural elements or struts.

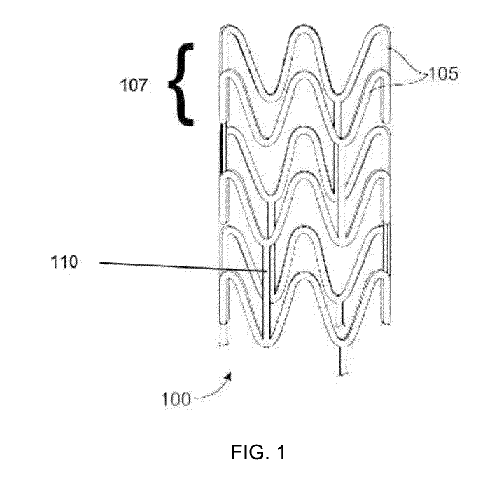

FIG. 1 depicts a view of an exemplary stent 100. In some embodiments, a stent may include a body, backbone, or scaffold having a pattern or network of interconnecting structural elements 105. Stent 100 may be formed from a tube (not shown). FIG. 1 illustrates features that are typical to many stent patterns including undulating sinusoidal cylindrical rings 107 connected by linking elements 110. As mentioned above, the cylindrical rings are load bearing in that they provide radially directed force to support the walls of a vessel. The linking elements generally function to hold the cylindrical rings together. A structure such as stent 100 having a plurality of structural elements may be referred to as a stent scaffold or scaffold. Although the scaffold may further include a coating, it is the scaffold structure that is the load bearing structure that is responsible for supporting lumen walls once the scaffold is expanded in a lumen.

The structural pattern in FIG. 1 is merely exemplary and serves to illustrate the basic structure and features of a stent pattern. A stent such as stent 100 may be fabricated from a polymeric tube or a sheet by rolling and bonding the sheet to form the tube. A tube or sheet can be formed by extrusion or injection molding. A stent pattern, such as the one pictured in FIG. 1, can be formed on a tube or sheet with a technique such as laser cutting or chemical etching. The stent can then be crimped on to a balloon or catheter for delivery into a bodily lumen. Alternatively, the scaffold design may be composed of radial bands that slide to increase the diameter of the scaffold. Such a design utilizes a locking mechanism to fix the stent at a target diameter and to achieve final radial strength. In other embodiments, the scaffold design could be braided polymer filaments or fibers.

The treatment methods disclosed herein can apply to bioresorbable scaffolds for both coronary and peripheral treatment. Bioresorbable polymer scaffolds for coronary artery treatment can have a length between 12 to 18 mm. Such coronary scaffolds may be laser cut from polymer tubes with a diameter between 2.5 mm to 4.5 mm and with a thickness/width of 140-160 microns.

The coronary scaffold may be configured for being deployed by a non-compliant or semi-compliant balloon from about a 1.1 to 1.5 mm diameter (e.g., 1.35 mm) crimped profile. Exemplary balloon sizes include 2.5 mm, 3.0 mm, 3.5 mm, and 4.0 mm, where the balloon size refers to a nominal inflated diameter of the balloon. The scaffold may be deployed to a diameter of between 2.5 mm and 5 mm, 2.5 to 4.5 mm, or any value between and including the endpoints. The pressure of the balloon to deploy the scaffold may be 12 to 20 psi. Embodiments of the invention include the scaffold in a crimped diameter over and in contact with a deflated catheter balloon.

The intended deployment diameter may correspond to, but is not limited to, the nominal deployment diameter of a catheter balloon which is configured to expand the scaffold. The balloon pressure and the diameter to which the balloon inflates and expands the scaffold may vary from deployment to deployment. For example, the balloon may expand the scaffold in a range between the nominal inflated diameter to the nominal inflated diameter plus 0.5 mm, e.g., a 3.0 mm balloon may expand a scaffold between 3 and 3.5 mm. In any case, the inflated diameter at deployment is less than the rated burst diameter of the balloon.

A scaffold may be laser cut from a tube (i.e., a pre-cut tube) that is less than an intended deployment diameter. In this case, the pre-cut tube diameter may be 0.7 to 1 times the intended deployment diameter or any value or range in between and including the endpoints.

Compared with bare metal stents, drug-eluting stents (DES) that are not bioresorbable have been shown to be safe and to result in greater absolute reductions in target lesion revascularization (TLR) and target vessel revascularization. A DES refers to a stent including a support structure (e.g., scaffold) and also includes a drug eluting coating over the support structure. The coating can include a polymer and a drug. The polymer functions as a drug reservoir for delivery of the drug to a vessel. The polymer can be non-biodegradable or bioresorbable.—The DES that are not bioresorbable include a metal support structure with a drug eluting coating

The ABSORB Bioresorbable everolimus eluting vascular scaffold (ABSORB BVS) of Abbott Vascular Inc. of Santa Clara, Calif. was recently developed to provide an approach to treating coronary artery lesions with transient vessel support and drug delivery. Preclinical evaluation in an animal model demonstrated substantial polymer degradation at 2-years post ABSORB BVS implantation, with complete disappearance of the BVS strut “footprint” in the vessel wall within a 3-4 year period. The first generation BVS (BVS revision 1.0) was tested in the ABSORB cohort A trial and demonstrated promising results with a low event clinical rate at up to 4 years follow up (EuroIntervention 2012; 7:1060-1061). The device was however limited by a slightly higher acute recoil compared to conventional metallic platform stents. The ABSORB Cohort A 5 year follow-up clinical results are shown in Table 1 below.

TABLE 1
5 year follow-up clinical results for ABSORB cohort A.

| | 6 Months | 12 Months | 4 Years | 5 Years |
|---|---|---|---|---|
| Hierarchical | 30 Patients | 29 Patients* | 29 Patients* | 29 Patients* |
| Ischemia | 3.3% (1)** | 3.4% (1)** | 3.4% (1)** | 3.4% (1)** |
| Driven MACE, | | | | |
| % (n) | | | | |
| Cardiac Death, | 0.0% | 0.0% | 0.0% | 0.0% |
| % | | | | |
| MI, % (n) | | | | |
| Q-Wave MI | 0.0% | 0.0% | 0.0% | 0.0% |
| Non Q-Wave | 3.3% (1)** | 3.4% (1)** | 3.4% (1)** | 3.4% (1)** |
| MI | | | | |
| Ischemia | | | | |
| Driven TLR, % | | | | |
| by PCI | 0.0% | 0.0% | 0.0% | 0.0% |
| by CABG | 0.0% | 0.0% | 0.0% | 0.0% |

No new MACE events between 6 months and 5 years
No scaffold thrombosis up to 5 years
*One patient withdrew consent after 6 months
**This patient also underwent a TLR, not qualified as ID-TLR (DS = 42%) followed by post-procedural troponin qualified as non Q MI and died from his Hodgkin's disease at 686 days post-procedure.

Improvements in design were therefore introduced in the second generation BVS (BVS revision 1.1), notably an enhanced mechanical strength, more durable support to the vessel wall, a reduced maximum circular unsupported surface area and a more uniform strut distribution and drug delivery. The performance of the next generation BVS revision 1.1 was subsequently investigated in the ABSORB Cohort B Trial which reported excellent clinical results at 1 and 2 year follow-up (J Am Coll Cardiol. 2011; 58: B66).

The polymer backbone is made of poly(L-lactide). The diameter of the scaffold is 3 mm and the length is 18 mm. The struts have a width of about 165 microns and thickness of about 152 microns. The coating is a mixture of poly(DL-lactide) and everolimus with a 1:1 ratio of polymer to drug. The coating is about 2 to 2.5 microns in thickness. The drug dose density is 100 μg/cm², which is the drug mass per scaffold surface area. The surface area of the scaffold is 160 mm², so the target drug dose is about 160 μg. The surface area of the scaffold per unit scaffold length is about 8.9 mm²/mm.

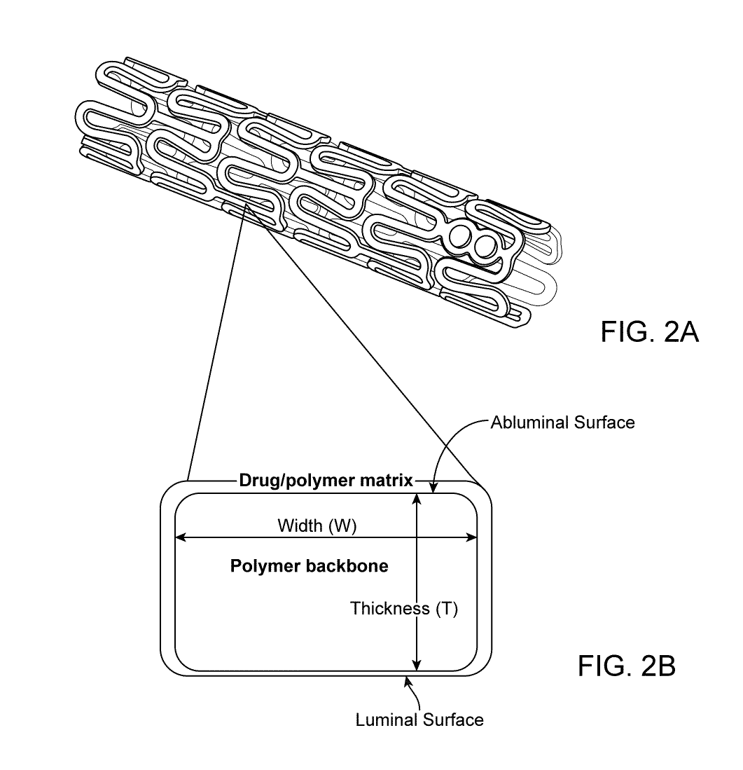

FIGS. 2A-B depicts the BVS revision 1.1 scaffold. FIG. 2A shows the scaffold in a crimped configuration. FIG. 2B show a cross-selection of a strut showing the polymer backbone or core of the strut surrounded by a drug/polymer matrix. The cross-section of the strut has an abluminal surface or side that faces the vessel wall and a luminal surface or side that faces the lumen of the vessel. The strut cross-section shown is rectangular with rounded corners with a width (W) and thickness (T). The BVS revision 1.1 scaffold is approximately square with an aspect ratio T/W close to 1.

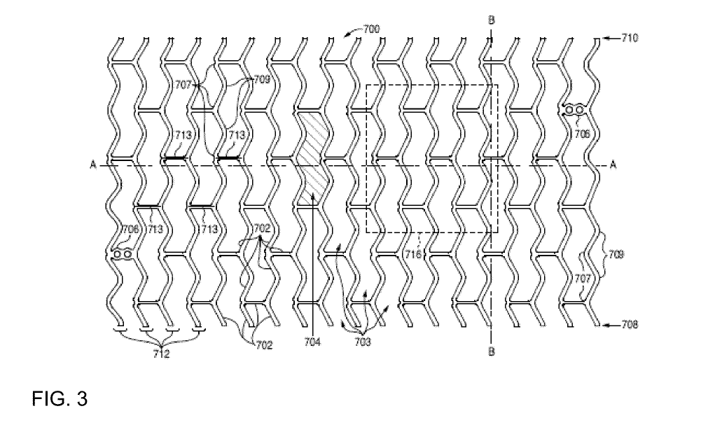

In a preferred embodiment a scaffold for coronary applications has the stent pattern described in U.S. application Ser. No. 12/447,758 (US 2010/0004735) to Yang & Jow, et al. Other examples of stent patterns suitable for PLLA are found in US 2008/0275537. FIG. 3 depicts exemplary stent pattern 700 from US 2008/0275537. The stent pattern 700 is shown in a planar or flattened view for ease of illustration and clarity, although the stent pattern 700 on a stent actually extends around the stent so that line A-A is parallel or substantially parallel to the central axis of the stent. The pattern 700 is illustrated with a bottom edge 708 and a top edge 710. On a stent, the bottom edge 708 meets the top edge 710 so that line B-B forms a circle around the stent. In this way, the stent pattern 700 forms sinusoidal hoops or rings 712 that include a group of struts arranged circumferentially. The rings 712 include a series of crests 707 and troughs 709 that alternate with each other. The sinusoidal variation of the rings 712 occurs primarily in the axial direction, not in the radial direction. That is, all points on the outer surface of each ring 712 are at the same or substantially the same radial distance away from the central axis of the stent.

The stent pattern 700 includes various struts 702 oriented in different directions and gaps 703 between the struts. Each gap 703 and the struts 702 immediately surrounding the gap 703 define a closed cell 704. At the proximal and distal ends of the stent, a strut 706 includes depressions, blind holes, or through holes adapted to hold a radiopaque marker that allows the position of the stent inside of a patient to be determined.

One of the cells 704 is shown with cross-hatch lines to illustrate the shape and size of the cells. In the illustrated embodiment, all the cells 704 have the same size and shape. In other embodiments, the cells 704 may vary in shape and size.

Still referring to FIG. 3, the rings 712 are connected to each other by another group of struts that have individual lengthwise axes 713 parallel or substantially parallel to line A-A. The rings 712 are capable of being collapsed to a smaller diameter during crimping and expanded to their original diameter or to a larger diameter during deployment in a vessel. Specifically, pattern 700 includes a plurality of hinge elements 731, 732, 733, 734. When the diameter of a stent having stent pattern 700 is reduced or crimped, the angles at the hinge elements decrease which allow the diameter to decrease. The decrease in the angles results in a decrease in the surface area of the gaps 703. In general, for most coronary applications, the diameter of the scaffold is 2 to 5 mm, or more narrowly 2.5 to 3.5 mm. In general, the length of the scaffold is 8 to 38 mm, or more narrowly, 8 to 12 mm, 12 to 18 mm, 18 mm to 38 mm. The scaffold for may be configured for being deployed by a non-compliant balloon, e.g., 2.5 to 4 mm diameter, from about a 1.8 to 2.2 mm diameter (e.g., 2 mm) crimped profile. The coronary scaffold may be deployed to a diameter of between about 2.5 mm and 4 mm. The present application includes results and analysis from the ABSORB Cohort B Trial. The studies were divided into two groups, Group B1 (N=45 patients) and Group B2 (N=56 patients) and each included QCA, IVUS, OCT, and IVUS VH. The follow-ups are as follows: Group B1-6 months, 18 months, and 24 months and Group B2-12 months, 18 months, and 36 months. Baseline Demographics and the lesion characteristics/acute success for the ABSORB Cohort B trial are shown in Tables 2 and 3.

TABLE 2
Baseline demographics of the ABSORB Cohort B trial.

| | Group 1 & 2 |
|---|---|
| | n = 101 |
| Male (%) | 72 |
| Mean age (years) | 62 |
| Previous MI (%) | 25 |
| Prior Cardiac Intervention on Target Vessel | 6 |
| (%) | |
| Diabetes mellitus (%) | 17 |
| Hypercholesterolemia req. med. (%) | 78 |
| Hypertension req. med. (%) | 62 |
| Current smoker (%) | 17 |

TABLE 3
Lesion characteristics/acute success for the ABSORB B trial.

| | N = 101 |
|---|---|
| | N_{Lesions} = 102 |
| Location of lesion (%) | |
| LAD | 43 |
| RCA | 33 |
| LCX | 23 |
| Ramus | 1 |
| Lesion classification (%) | |
| A | 1 |
| B1 | 55 |
| B2 | 40 |
| C | 4 |
| Clinical Device success (%) | 100 |
| Clinical Procedure success (%) | 98 |

Clinical Device Sucess = Successful delivery & deployment of the BVS at intended target lesion & successful withdrawal of the BVS delivery system w/ attainment of final resisdual stenosis of less than 50% of the target lesion by QCA (by visual estimation if QCA unavailable). Standard pre-dilation catheters & post-dilation catheters (if applicable) may be used. Bailout patients will be include as device success only if the above criteria for clinical device are met.
Clinical Procedure Success = Same as definition above and/or using any adjunctive device without occurrence of ischemia driven major adverse cardiac event (MACE) during the hospital stay w/ a maximum of first seven days post index procedure.

Edge Effects

The vascular response of the segments adjacent to the proximal and distal edges of the ABSORB Everolimus-Eluting Bioresorbable Vascular Scaffold were investigated at 6 Months and 1 year follow-up. JACC Cardiovasc Interv. 2012 June; 5(6):656-65. Results are based on a virtual histology intravascular ultrasound study.

The study sought to investigate in vivo the vascular response at the proximal and distal edges of the ABSORB everolimus-eluting bioresorbable vascular scaffold (BVS). The edge vascular response after implantation of the BVS has not been previously investigated.

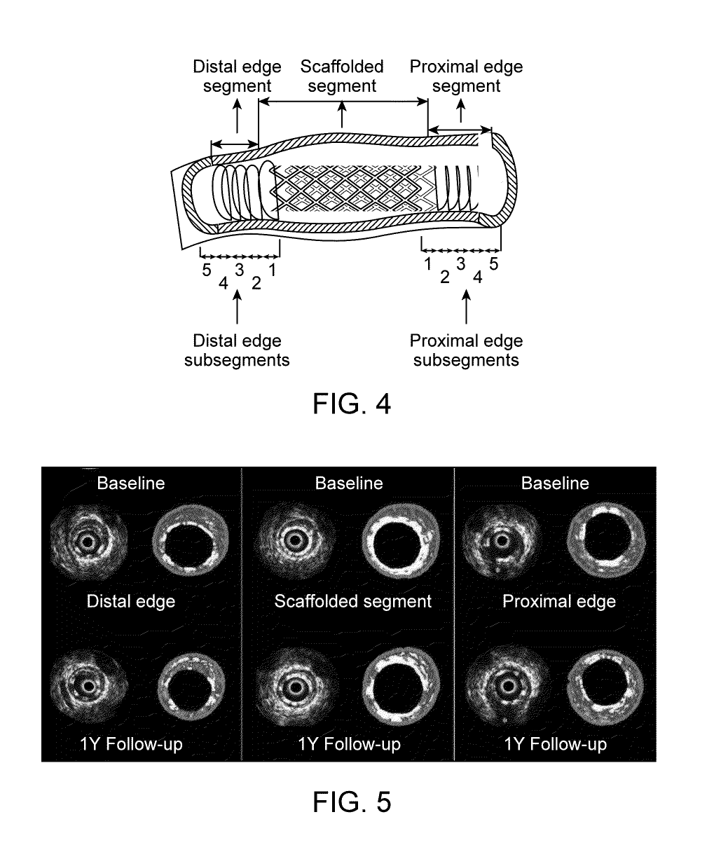

FIG. 4 depicts a schematic view of a scaffold deployed in a vessel segment showing a scaffolded segment, a proximal edge segment, and a distal edge segment. The scaffold extends along a longitudinal axis of the vessel segment and supports the segment through contact with the wall of the vessel between a proximal end of the scaffold and a distal end of the scaffold. The proximal edge segment is proximally adjacent to the proximal end of the scaffold and is not supported directly through contact with the scaffold. The distal edge segment is distally adjacent to the distal end of the scaffold and is not supported directly through contact with the scaffold. The proximal and distal edge segments are each divided into five subsegments.

The adjacent (5-mm) proximal and distal vessel segments to the implanted ABSORB BVS were investigated at either 6 months (B1) or 1 year (B2) with virtual histology intravascular ultrasound (VH-IVUS) imaging. At the 5-mm proximal edge, the only significant change was modest constrictive remodeling at 6 months. The constrictive remodeling is demonstrated by a decrease in the vessel cross sectional area.

The change in vessel cross-sectional area at 6 months from deployment is −1.80% [−3.18; 1.30], p<0.05). There was a tendency for the constrictive remodeling to regress or decrease after 6 months, since at 1 year the change vessel cross-sectional area since deployment is −1.53% [−7.74; 2.48], p=0.06).

The relative change of the fibrotic and fibrofatty (FF) tissue areas at the proximal segment were not statistically significant at either time point. At the 5-mm distal edge, a significant increase in the FF tissue of 43.32% [−19.90; 244.28], (p<0.05) 1-year post-implantation was evident. The increase may be at least 40%. The changes in dense calcium need to be interpreted with caution since the polymeric struts are detected as “pseudo” dense calcium structures with the VH-IVUS imaging modality.

FIG. 5 depicts VH-IVUS images of the distal edge segment, scaffolded segment, and the proximal edge segment at baseline and 1 year follow-up.

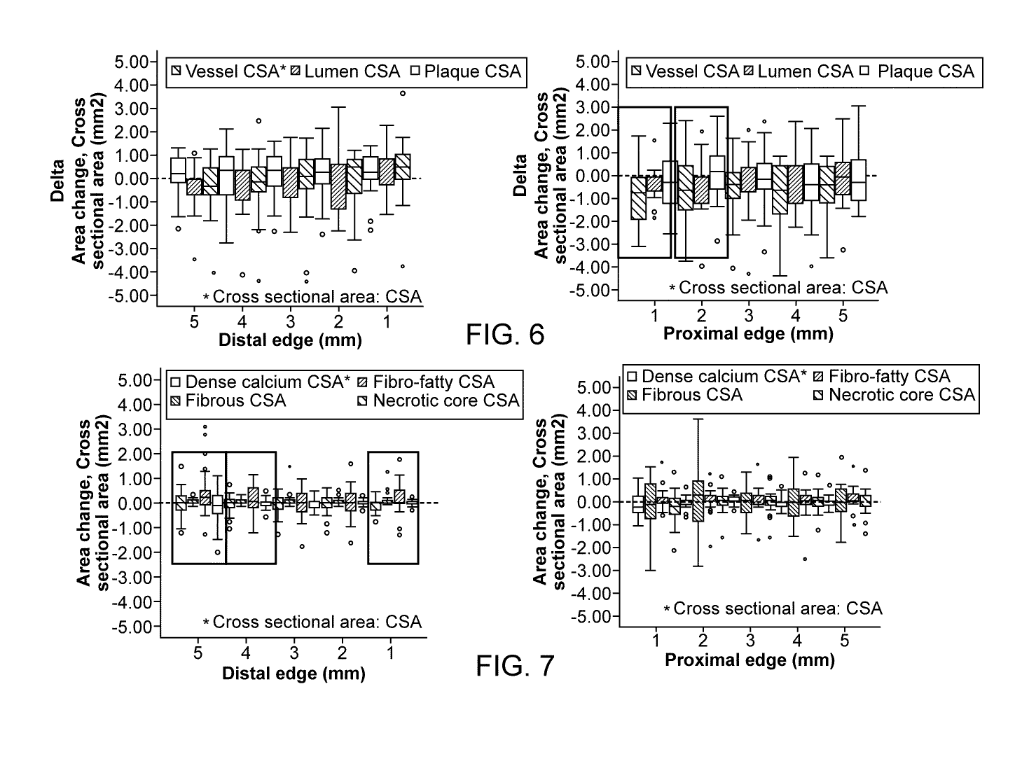

FIG. 6 depicts the change in vessel, lumen, and plaque cross sectional area along the distal edge and the proximal edge at 1 year follow-up. Constrictive remodeling is evident in the proximal edge at 1 year only at 1 and 2 mm and has disappeared at 5 mm.

FIG. 7 depicts the tissue composition along the distal edge and the proximal edge at 1 year follow-up. The average change in cross sectional area of dense calcium, fibrous, fibro-fatty, and necrotic core is shown. At the distal edge, an increase in fibro-fatty component is evident at 1, 3, and 4 mm.

The vascular response up to 1 year after implantation of the ABSORB BVS demonstrated some degree of proximal edge constrictive remodeling that tends to regress at 1 year. Some degree of proximal edge and distal edge plaque compositional changes were observed with increase of the fibrofatty tissue component at 1-year. The distal edge increases in fibro-fatty tissue resulting in nonsignificant plaque progression with adaptive expansive remodeling. This morphological and tissue composition behavior appears to not significantly differ from the behavior of metallic drug-eluting stents at the same observational time points. The constrictive remodeling at the proximal edge tends to regress at 1-year. This biological behavior is similar to that observed with the metallic devices at the same follow-up points.

Tables 4A and 4B provide the proximal edge vascular response in terms of percent change in vessel cross-sectional area (CSA), lumen CSA, and plaque CSA. Tables 5A and 5B provide the distal edge vascular response.

TABLE 4A
Proximal edge vascular response.

| Proximal edge segment, | Vessel CSA | Lumen CSA | Plaque CSA |
|---|---|---|---|
| (%) change | (mm²) | (mm²) | (mm²) |
| 6-months (n = 23) | −1.80 | −4.10 | −4.04 |
| | [−3.18; 1.30] | [−11.61; 8.79] | [−10.65; 11.05] |
| p-value | <0.05 | NS | NS |
| 12-months (n = 25) | −1.83 | −5.32 | −2.03 |
| | [−7.74; 2.48] | [−12.36; 4.24] | [−8.39; 7.76] |
| p-value | NS | NS | NS |

TABLE 4B
Proximal edge vascular response.
Time after the imaging procedure

| | 6-months (n = 23) | | | 1-year (n = 25) | | |
|---|---|---|---|---|---|---|
| Proximal Edge | Vessel CSA (mm²) | Lumen CSA (mm²) | Plaque CSA (mm²) | Vessel CSA (mm²) | Lumen CSA (mm²) | Plaque CSA (mm²) |
| Baseline | 13.20 | 7.15 | 5.88 | 13.89 | 2.25 | 7.02 |
| | [10.81; 1 [symbol] .90] | [ [symbol] .80; 8.65] | [4.22; 7.08] | [12.55; 17.24] | [6.44; 8.40] | [8.52; 7.80] |
| Follow-Up | 13.38 | 7.15 | 5.49 | 13.71 | [symbol] .0 | 7.08 |
| | [10.26; 15. [symbol] 9] | [ [symbol] .60; 8.41] | [3. [symbol] 6; 7.25] | [12.22; 36.12] | [6. [symbol] ; 8.30] | [5.36; 8.38] |
| Median Absolute | −0.25 | −0.27 | 0.25 | −0.19 | −0.35 | −0.1 [symbol] |
| Difference | [−0.54; 0.18] | [−0.78; 0.67] | [−0.63; 0.60] | [−1.06; 0.33] | [−0.7 [symbol] ; 0.21] | [−0.59; 0.37] |
| p-value | <0.05 | NS | NS | NS | NS | NS |

[symbol]

indicates data missing or illegible when filed

TABLE 5A
Distal edge vascular response.

| Distal edge segment, | Vessel CSA | Lumen CSA | Plaque CSA |
|---|---|---|---|
| (%) change | (mm²) | (mm²) | (mm²) |
| 6-months (n = 18) | −0.59 | −0.32 | 7.0 |
| | [−3.74; 7.19] | [−7.71; 7.20] | [−11.97; 18.36] |
| p-value | NS | NS | NS |
| 12-months (n = 30) | 3.45 | 0.95 | 5.73 |
| | [−2.08; 6.91] | [−7.56; 7.48] | [−6.49; 25.47] |
| p-value | NS | NS | NS |

TABLE 5B
Distal edge vascular response.
Time after the imaging procedure

| | 6-months (n = 18) | | | 1-year (n = 30) | | |
|---|---|---|---|---|---|---|
| Distal Edge | Vessel CSA (mm²) | Lumen CSA (mm²) | Plaque CSA (mm²) | Vessel CSA (mm²) | Lumen CSA (mm²) | Plaque CSA (mm²) |
| Baseline | 12.79 | 7.27 | 7.02 | 10.28 | 6.70 | 4.47 |
| | [10.17; 16.38] | [5.62; 7.90] | [4.15; 7.89] | [9.13; 13.46] | [5.83; 7.80] | [2.29; 5.6 [symbol] ] |
| Follow-Up | 13.87 | 6.8 [symbol] | 6.07 | 10.40 | 6.76 | 4.46 |
| | [10.42; 15.15] | [6.11; 8.44] | [4.90; 8.40] | [9.88; 13.33] | [5.56; 7.78] | [3.20; 6.61] |
| Median Absolute | −0.07 | −0.03 | 0.35 | 0.40 | 0.09 | 0.27 |
| Difference | [−0.51; 1.00] | [−0.55; 0.58] | [−0.82; 0.97] | [−0.26; 0.63] | [−0.49; 0.43] | [−0.27; 0.97] |
| p-value | NS | NS | NS | NS | NS | NS |

[symbol]

indicates data missing or illegible when filed

Table 6 shows ABSORB Cohort B trial results up to 2 years follow-up. Table 6 shows no scaffold thrombosis out to 2 years and only 2 additional TLR events between 1 year and 2 years, and MACE rate of 8.9% (3 non-Q wave MI, 6 ID TLR) at 2 years which is comparable to Xience V.

TABLE 6
Clinical results at 2 year follow-up of Groups
1 and 2 for of ABSORB B trial.

| | 30 Days | 6 Months | 12 Months | 2 Years |
|---|---|---|---|---|
| Non-Hierarchical | N = 101 | N = 101 | N = 101 | N = 100* |
| Cardiac Death % | 0 | 0 | 0 | 0 |
| Myocardial Infarction % (n) | 2.0 (2) | 3.0 (3) | 3.0 (3) | 3.0 (3) |
| Q-wave MI | 0 | 0 | 0 | 0 |
| Non Q-wave MI | 2.0 (2) | 3.0 (3) | 3.0 (3) | 3.0 (3) |
| Ischemia driven TLR % (n) | 0 | 2.0 (2) | 4.0 (4) | 6.0 (5) |
| CABG | 0 | 0 | 0 | 0 |
| PCI | 0 | 2.0 (2) | 4.0 (4) | 6.0 (6) |
| Hierarchical MACE % (n) | 2.0 (2) | 5.0 (5) | 6.9 (7) | 9.0 (9) |
| Hierarchical TVF % (n) | 2.0 (2) | 5.0 (5) | 6.9 (7) | 9.0 (9) |

*One patient missed the 2-year FUP
No scaffold thrombosis by ARC or Protocol
MACE: Cardiac death, MI, ischemia-driven TLR
TVF: Cardiac death, MI, ischemia-driven TLR, ischemia-driven TVR

Compliance

Vascular compliance changes in the coronary vessel wall after bioresorbable vascular implantation in the treated and adjacent segments. Implantation of a metallic prosthesis creates local stiffness with a subsequent mismatch in compliance between the scaffolded and the immediate adjacent segments.

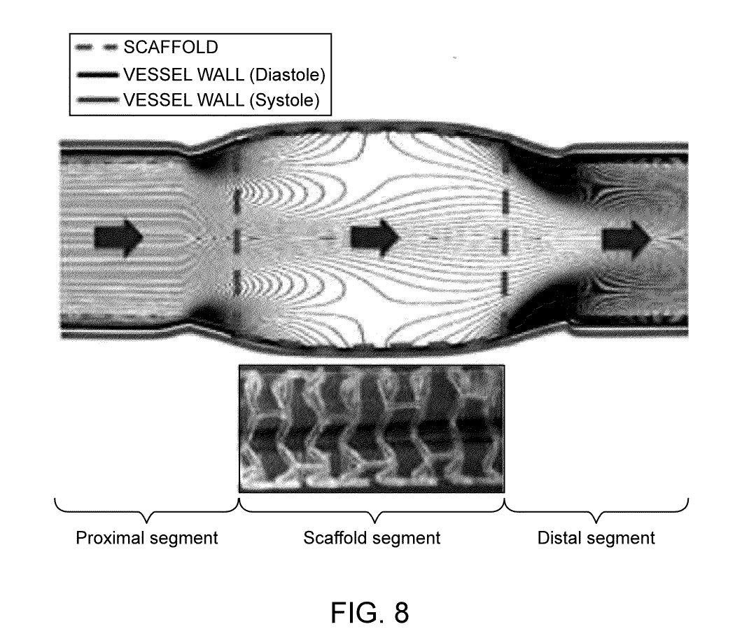

FIG. 8 shows a schematic of a cross section of a scaffold deployed in a vessel showing a scaffolded segment, a proximal segment, and a distal segment. The direction of flood flow is shown by arrows and streamlines of blood flow are also shown. This process may potentially create disturbances in flow and heterogeneous distribution of wall shear stress with subsequent risk of stent thrombosis or restenosis. Bioresorbable ABSORB scaffolds (Generation 1.0 and 1.1, tested in ABSORB Cohort A and Cohort B trials respectively) made of polylactide have less stiffness compared to metallic platform stents and are completely bioresorbed in the long-term. The mismatch in vascular compliance after ABSORB scaffold implantation and its long-term resolution with bioresorption was analyzed.

A total of 83 patients from the ABSORB trials underwent palpography investigations (30 and 53 patients from ABSORB Cohort A and B, respectively) to measure the compliance of the scaffolded and adjacent segments at various time points (from pre-implantation up to 24 months). The mean of the maximum strain values in all cross sections was calculated per segment by utilizing the Rotterdam Classification (ROC) score and expressed as ROC/mm.

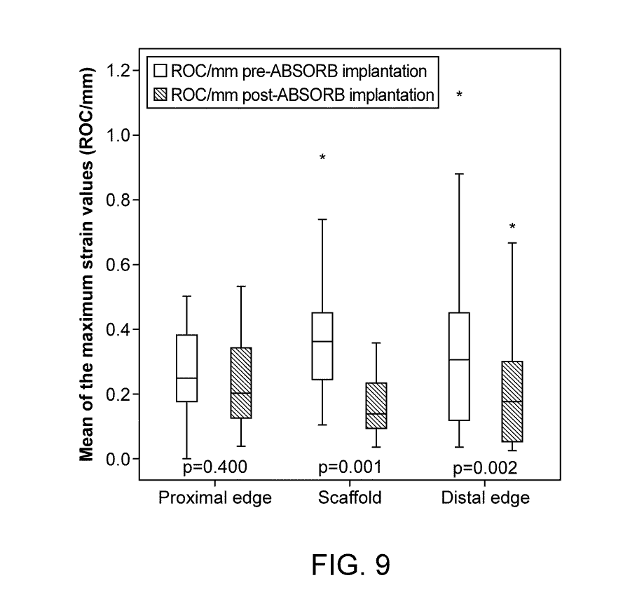

FIG. 9 depicts the mean of the maximum strain values for each of the three segments. The results in FIG. 9 show that scaffold implantation leads to a significant decrease in vessel compliance (median [IQR]) at the scaffolded implantation segment (from 0.37[0.24-0.45] to 0.14[0.09-0.23], p<0.001).

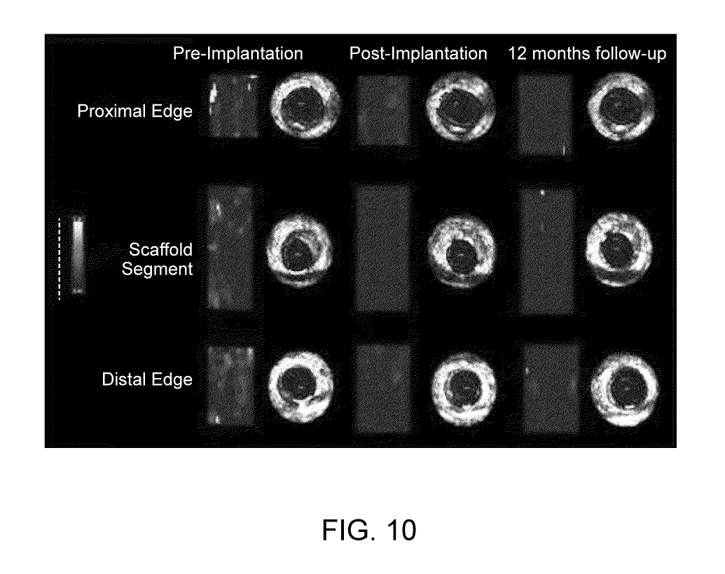

After scaffold implantation mismatch in compliance was evident in patients with paired analyses between the scaffolded and adjacent segments (proximal: 0.23[0.12-0.34], scaffold: 0.12[0.07-0.19], distal: 0.15[0.05-0.26], p=0.042). Thus, mismatch is greater between the scaffolded segment and the proximal segment and the scaffolded segment and the distal segment. The former may be at least 90% or 90 to 100% and the latter may be at least 10% or 10 to 40%. This reported compliance mismatch disappeared at short and mid-term follow-up (6 and 12 months). FIG. 10 depicts the compliance in each of the segments pre-implantation, post-implantation, and 1 year follow-up. In FIG. 10, darker is low compliance and lighter is high compliance.

The conclusions of the results are that the ABSORB scaffold decreases vascular compliance at the site of scaffold implantation. A compliance mismatch is present immediately post-implantation and in contrast to metallic stents disappears in the mid-term likely leading to a normalization of the rheological behavior of the scaffolded and adjacent segments. The Cohort A and B scaffolds have also been shown to exhibit low late loss and exhibit low restenosis. The BVS scaffolds provide these favorable clinical outcomes in spite of the thicker/wider struts of these scaffolds (approx. 150 microns) compared to metal stents, e.g., Xience V and Taxus Express.

It is believed that favorable clinical outcomes thus far for patients are due to synergy between various unique aspects of the BVS scaffolds:
1) The BVS scaffolds are stiff and strong enough to support the vessel wall at a required diameter with low recoil, but are flexible enough to reduce trauma. Polymers are inherently less stiff and strong than metals, as discussed below. The processing of scaffold, as described below, increases the stiffness and strength of the polymer of the scaffold to provide necessary material and scaffold properties, i.e., strength, stiffness, radial stiffness, and radial strength. The processing reduces the deficiency of lower strength and stiffness of polymer as compared to metals. However, the processed material and scaffold are still less stiff than metals, reducing edge effects and thrombosis.
2) The degradation properties of the scaffold is also designed to provide the necessary strength and stiffness for a sufficient period of time to allow remodeling, described in detail below. After this time, the scaffold's properties deteriorate and the support is transferred to the remodeled vessel gradually. This gradually eliminates compliance mismatch, resulting in reduced flow distribution with reduced turbulence, which reduces risk of thrombosis. The fast disappearance of the scaffold through absorption reduces risk of thrombosis.
3) The controlled deployment of the BVS scaffold performed in a controlled manner may also contribute to reduced thrombosis and reduced thrombosis. A bioresorbable scaffold or a balloon expandable metallic stents is crimped to a reduced diameter over a deflated balloon. When the crimped stent is positioned at an implant site, the stent or scaffold is deployed at the treatment site by inflation of the balloon. The inflation of the balloon expands the stent or scaffold at the implant site. The balloon is then deflated and withdrawn from the patient. The inflation of the balloon that deploys the scaffold is performed slower than is typically used for deploying a metal stent. Fast inflation rates result in a balloon inflating first at the edges and then propagating to center, resulting in a dog-bone or tapered structure of the balloon and the stent. Slower inflation rates result in more uniform deployment (less edge taper) along the length of the scaffold, which reduces thrombosis risk.
4) The inflation rate of the BVS scaffold, which is recommended to be 6 psi/s or less, also results in less trauma to the vessel wall, potentially resulting in lower restenosis and thrombosis.
5) The scaffold pattern, described herein, is also designed to provide the necessary radial strength, radial stiffness, and flexibility. Thus, pattern takes advantage of the material properties modification described above.
6) Higher drug doSE due to thicker struts of BVS scaffolds may contribute to low thrombosis and restenosis.

In general, the treatment with bioabsorbable polymer stents has a number of advantages over permanent implants: (i) The stent disappears from the treated site resulting in reduction or elimination of late stent thrombosis; (ii) disappearance of the stent facilitates repeat treatments (surgical or percutaneous) to the same site; (iii) disappearance of the stent allows restoration of vasomotion at the treatment site (the presence of a rigid permanent metal stent restricts vasomotion); (iv) the bioabsorbability results in freedom from side-branch obstruction by struts; (v) the disappearance results in freedom from strut fracture and ensuing restenosis. Some of these advantages may be relevant to improving clinical outcomes for non-diabetic and diabetic patients.

In the short term and over the long term, a bioresorbable scaffold has the advantage of being less traumatic to the vessel wall. Since the bioresorbable scaffold degrades with time and eventually disappears, trauma associated with the presence of a scaffold decreases with time and eventually disappears. Resorption of a bioresorbable scaffold which restores vasomotion of the vessel wall may reduce long term thrombotic risk.

The thrombogenic potential has been evaluated based on platelet adhesion to the BVS cohort B scaffold deployed ex vivo. Platelets are indispensable initiators of thrombosis and their adhesion to intravascular devices is the critical step in the thrombus formation. In a study of platelet adhesion, metallic coronary stents (BMS Multilink Vision and Xience V) and BVS scaffolds were deployed in a Chandler Loop perfused with freshly prepare porcine platelet rich plasma (PRP) instead of whole blood. The extent of platelet adhesion is determined by measuring the LDH activity extracted from the adherent platelets which is directly proportional to the number of platelets. Such properties may be of particular benefit in diabetic vascular disease. Thrombogenicity based on the adhesion of platelets was consistently the highest for the BMS Multi-Link Vision followed by the Xience V stents and followed by the BVS scaffolds.

Thicker scaffold struts with a higher total dose of drug may be beneficial in reducing incidence of smooth muscle cell proliferation. The thicker struts in the BVS scaffold, about 150 to 165 microns, results in a total dose of everolimus that is almost two fold higher than XIENCE V.

One aspect is the use of a polymer, in particular a bioresorbable polymer, for the scaffold. A polymer scaffold may be less traumatic to a vasculature. Polymers are softer, less stiff or have a lower modulus than metals. Thus, the presence of a softer, more flexible implant may be less traumatic to a soft, flexible vessel segment than a metal implant. For example, aliphatic bioresorbable polymers have tensile moduli generally less than 7 GPa and in the range of 2 to 7 GPa (US2009/0182415). Poly(L-lactide) has a tensile modulus of about 3 GPa.

Metals used to make a stent and their approximate moduli include stainless steel 316L (143 GPa), tantalum (186 GPa), Nitinol or nickel-titanium alloy (83 Gpa), and cobalt chromium alloys (243 Gpa). These moduli are significantly higher than aliphatic polymers. The strengths of these metals are also significantly higher than the polymers as well. As a result, a bioresorbable polymeric scaffold has thicker struts to help compensate for the difference in the material properties to provide a radial stiffness and radial strength this sufficient to provide patency.

Also, the mismatch of the properties of a polymer scaffold and a vessel segment is lower than for a metallic scaffold. This mismatch can be expressed formally in terms of compliance mismatch between the scaffold and the vessel segment at the implant site. The compliance of a material, which is the inverse of stiffness or modulus of a material, refers to the strain of an elastic body expressed as a function of the force producing the strain. The compliance of a scaffold or radial compliance of the scaffold can likewise be defined as the inverse of the radial stiffness of the scaffold. The radial stiffness of the bioresorbable scaffold is lower than a metallic scaffold, so the radial compliance of the bioresorbable scaffold is higher than a metallic scaffold. The compliance mismatch of a polymer scaffold is lower than a metallic stent.

The compliance of a stent, both nondegradable and resorbable, is necessarily much lower than the vessel segment in order for the scaffold to support the vessel at a deployed diameter with minimal periodic recoil due to inward radial forces from the vessel walls. Additionally, it results in better conformity (and less straightening) of the scaffolded segment to the overall curvature of the adjacent segments in the treated vessel. However, an additional aspect of a bioresorbable polymer scaffold that may contribute to favorable clinical outcomes is that the compliance mismatch decreases with time due to the degradation of the bioresorbable polymer. As the polymer of the scaffold degrades, mechanical properties of the polymer such as strength and stiffness decrease and compliance increases. As a result, the radial strength of the scaffold decreases with time and the compliance of the scaffold increases with time since these properties depend on the properties of the scaffold material.

In the long term, the compliance of a vessel segment with an implanted scaffold converges to that of the natural compliance of the vessel. The convergence of the compliance occurs gradually as the vessel segment heals. Since natural compliance of a vessel segment is eventually restored due to complete resorption of the scaffold, natural vasomotion of the vessel segment is also restored. Compliance mismatch in the treatment with metallic stents is permanent and has been identified as a contributor to the process of restenosis and potentially late adverse events.

Another aspect that may contribute to favorable clinical outcomes of bioresorbable scaffolds is a higher drug loading or target dose of the bioresorbable scaffold. From above, the BVS scaffold in the ABSORB Cohort A and B trials is 18 mm long and has a drug dose density of 100 μg/cm² and a target drug dose of about 160 μg. The target drug dose per unit scaffold length of the ABSORB Cohort B trial scaffold is about 8.9 μg/mm. The delivery of the target dose to the vessel can occur over a period of about 2 to 3 months after implantation.

The drug dose density of the XIENCE V® stent (http://www.accessdata.fda.gov/cdrh_docs/pdf11/P110019b.pdf) and TAXUS Express® (American Heart Journal Volume 163, Number 2, p. 143-148) are both reported to be 100 μg/cm². However, the BVS target dose and dose per unit length is larger due to the wider and thicker struts compared to these stents: XIENCE V® (91 mm×81 mm) and TAXUS Express® (91 mm×132 mm).

BMS and metallic DES stents typically have strut widths and thicknesses much less than the BVS stent (Interventional Cardiology, Vol. 6, Issue 2, pp. 143-147). The larger strut width and strut thickness, or equivalently, larger surface area of the BVS scaffold may also contribute to favorable clinical outcomes of diabetic patients. The larger strut width and strut thickness or surface area of a bioresorbable scaffold contributes by providing a higher target dose due to the higher surface area of contact with the vessel walls.

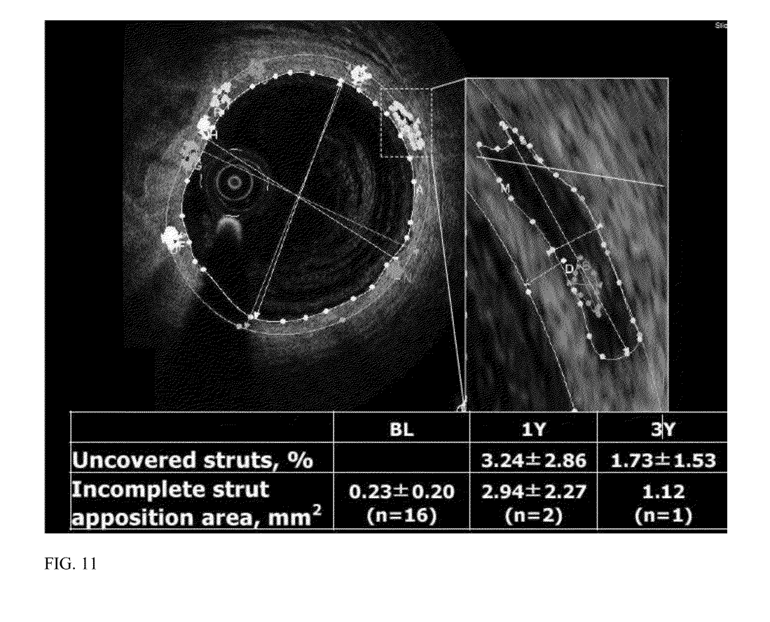

The 3 year results of the ABSORB Cohort B Trial are further provided summarized in FIGS. 11 to 25. These results include imaging and vasomotion data. It has previously been established in human testing that the mechanical integrity and the absence of recoil was maintained over a period of 6 months.

The clinical results for Cohort B Groups 1 and 2 are shown in Table 7. There is no scaffold thrombosis by ARC or protocol.

TABLE 7
Clinical results for Cohort B for Groups 1 and 2.

| | 30 Days | 6 Months | 12 Months | 2 Years | 3 Years |
|---|---|---|---|---|---|
| Non-Hierarchical | N = 101 | N = 101 | N = 101 | N = 100* | N = 100* |
| Cardiac Death % | 0 | 0 | 0 | 0 | 0 |
| Myocardial Infarction % (n) | 2.0 (2) | 3.0 (3) | 3.0 (3) | 3.0 (3) | 3.0 (3) |
| Q-wave MI | 0 | 0 | 0 | 0 | 0 |
| Non Q-wave MI | 2.0 (2) | 3.0 (3) | 3.0 (3) | 3.0 (3) | 3.0 (3) |
| Ischemia driven TLR % (n) | 0 | 2.0 (2) | 4.0 (4) | 6.0 (6) | 7.0 (7) |
| CABG | 0 | 0 | 0 | 0 | 0 |
| PCI | 0 | 2.0 (2) | 4.0 (4) | 6.0 (6) | 7.0 (7) |
| Hierarchical MACE % (n) | 2.0 (2) | 5.0 (5) | 6.9 (7) | 9.0 (9) | 10.0 (10) |
| Hierarchical TVF % (n) | 2.0 (2) | 5.0 (5) | 6.9 (7) | 11.0 (11) | 13.0 (13) |

*One patient missed the 2-year FUP
MACE: Cardiac death, MI, ischemia-driven TLR
TVF: Cardiac death, MI, ischemia-driven TLR, ischemia-driven TVR

The results include serial image acquisition at baseline, 1 year, and 3 years including events: OCT Optional 19 patients, IVUS-GS Mandatory 45 patients, IVUS-VH Mandatory 38 patients, IVUS-Echogenicity, derived from GS 29 patients, and angiography mandatory 51 patients.

In the following months (from 6 to 12 months) it has been shown that physiological and pharmacological vasomotion reappears confirming the fact that the mechanical stiffness of the polymer is progressively replaced by de novo formation of malleable tissue such as proteoglycan.

At 2 years it has been demonstrated that the scaffold device despite its malleable and deformable structure did not undergo any reduction in area or volume. In contrast, a late enlargement of the scaffold was documented, probably due to the intraluminal expansive force of the systolic/diastolic wall stress. This late enlargement of the scaffold compensates for the intraluminal growth of neointimal tissue.

The ultimate expectation of the bioabsorbable stent intervention is the occurrence of late lumen enlargement, associated with wall thinning, without expansive remodeling.

At 3-year follow-up of the Cohort B showed: stable late loss, return of vasomotion to the scaffolded segment, enlargement of scaffold area as well as mean lumen area despite persisting increase of neointima, reduction of plaque area, and bioresorption slower than the first generation of ABSORB (1.0).

3 year follow-up results show improvements in blood vessel movement, area inside the vessel, and reduction of plaque where the scaffold was placed.

At three years, the rate of major adverse cardiovascular events (MACE) in 101 patients was 10 percent, similar to a comparative set of data with a best-in-class drug eluting stent at three years. MACE is a combined endpoint that includes heart attacks, deaths for heart related causes or re-blockages of the blood vessel resulting in symptoms requiring the need for additional procedures at the original site of scaffold implantation.

In a subset of 46 patients, pictures inside the blood vessel using state-of-the art imaging techniques showed improvements in vessel motion and an average increase of 7.3 percent between one and three years in the area within the blood vessel, allowing more blood to flow through the vessel as the body requires, a finding unique to Absorb and not typically observed with metallic stents that cage the vessel. There was also a decrease of plaque inside the vessel between two and three years. Plaque is made up of fat, cholesterol, calcium and other deposits that accumulate on the inner wall of the artery in patients with coronary heart disease and can slow or stop blood flow to the heart.

The clinical data up to 3 years a showed an ID-MACE rate of 10.0% with no events of scaffold thrombosis. The late loss at 3 years was 0.32±042 mm. The IVUS grey scale results revealed scaffold and lumen enlargement between baseline and 3 years (6.29±0.91 vs. 7.08±1.55, p<0.0001 and 6.29±0.90 vs. 6.81±1.62, p=0.0155, respectively). The scaffold enlargement was confirmed by OCT (7.76±1.07 at baseline vs. 8.64±2.15 at 3 years, p=0.0446).

The IVUS-VH and the IVUS-derived echogenicity results show signs of bioresorption indicated by a significant reduction in dense calcium and in percent hyper-echogenic area, respectively, between baseline and 3 years.

FIG. 11 shows that the percent of struts uncovered by an endothelial layer decreases between 1 and 3 years from baseline. FIG. 11 also shows that the incomplete apposition area increases between baseline and 1 year and then decreases between 1 year and 3 years after baseline. The incomplete strut apposition area can decrease by at least 100%, 200%, 300%, or between 100 and 300%.

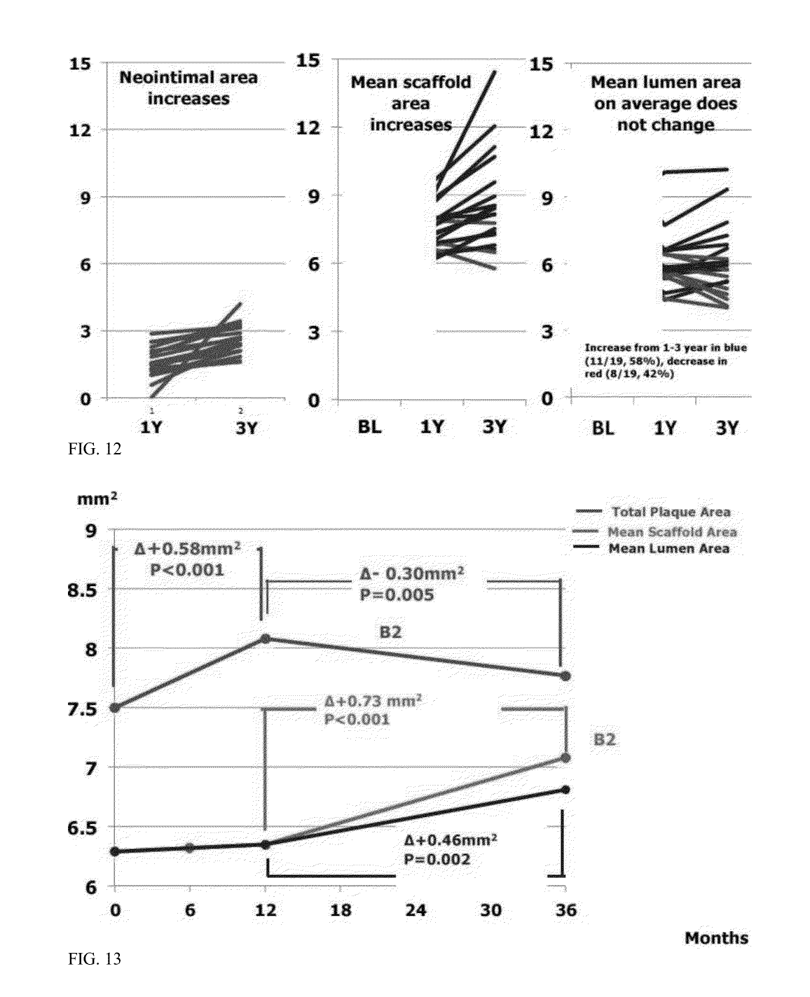

FIG. 12 depicts the neointimal area, mean scaffold area, and mean lumen area from OCT for 19 patients between 1 and 3 years follow-up. FIG. 12 shows that the neointimal area increases between 1 year and 3 years after baseline. The percentage increase can be at least 50%, at least 100%, at least 200%, at least 300%, or between 100% and 300%, or between 200% and 300%. FIG. 12 also shows that the mean scaffold area increases between 1 year and 3 years after baseline. The increase may be between 10% and 40%. FIG. 12 also shows the mean lumen area on average does not change significantly or is relatively constant between 1 year and 3 years after baseline. In particular, the mean lumen area may vary between change between 1 and 3 years by less than 20%, less than 10%, less than 5% or between 10 and 20%.

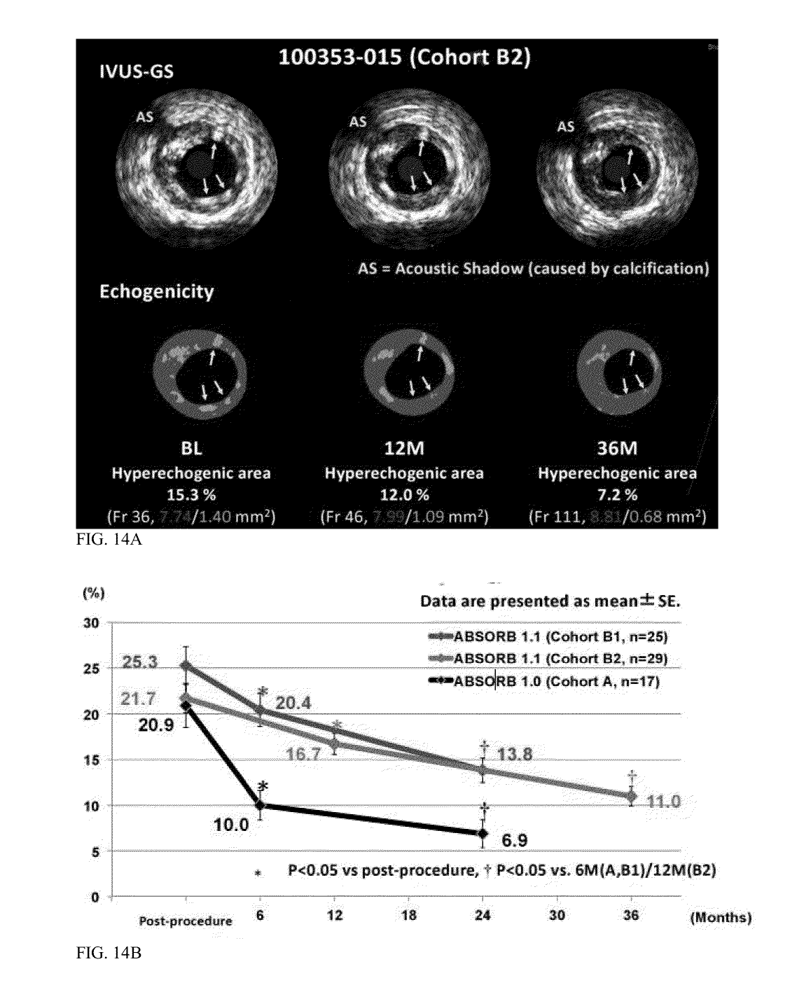

FIG. 13 depicts the serial quantitative IVUS analysis of the total plaque area (uppermost curve), mean scaffold area (middle curve), and mean lumen area (lowermost curve) for Group B2 between baseline and 3 years follow-up. The total plaque area increases between baseline and 6 months and between 6 months and 1 year and then decreases between 1 year 2 years and between 2 and 3 years. Both the mean scaffold area and the and the mean lumen area are relatively constant (e.g., vary by less than 2%) between baseline and 6 months and 6 months and 1 year and then increase between 1 year and 2 years and between 2 years and 3 years. The increase between 1 and 3 years may be 5 to 15%.

FIG. 14A shows the IVUS-GS and Echogenicity images for Group B2 at baseline, 1 year, and 3 years. FIG. 14A shows that that the hyperechogenic area decreases between baseline and 12 months (15.3% to 12%) and decreases further between 12 months and 3 years to 7.2%.

FIG. 14B depicts the percentage change in hyperechogenic area (HEA) for ABSORB 1.1, Cohorts B1 (uppermost curve) and B2 (middle curve), and ABSORB 1.0 Cohort A (bottom curve). As shown, the HEA for ABSORB 1.1 Cohorts or Groups decrease between baseline, 6 months, 1 year, 2 years, and 3 years. The HEA for ABSORB 1.1 decreases between baseline and 6 months and 24 months. The drop in the HEA between baseline and 6 months is more significant for ABSORB 1.0 than 1.1, about 50% compared to about 10% for 1.1.

Table 8 shows the VH results of dense calcium area percent at baseline, 1 year, and 3 years follow-up. The dense calcium area percent decreases between baseline and 1 year and between 1 year and 3 years.

TABLE 8
VH results of dense calcium area percent at baseline, 1 year, and 3 years.

| | BL | 12 month | 36 month | Difference | Difference | P values | P values |
|---|---|---|---|---|---|---|---|
| | n = 38 | n = 38 | n = 38 | 1 Y-3 Y | BL-3 Y | 1 Y-3 Y | BL-3 Y |
| Dense calcium | 30.3 | 24.8 | 21.8 | −3.0 ± 5.1 | −8.5 ± 9.6 | 0.0015 | <0.001 |
| area, % | | | | | | | |

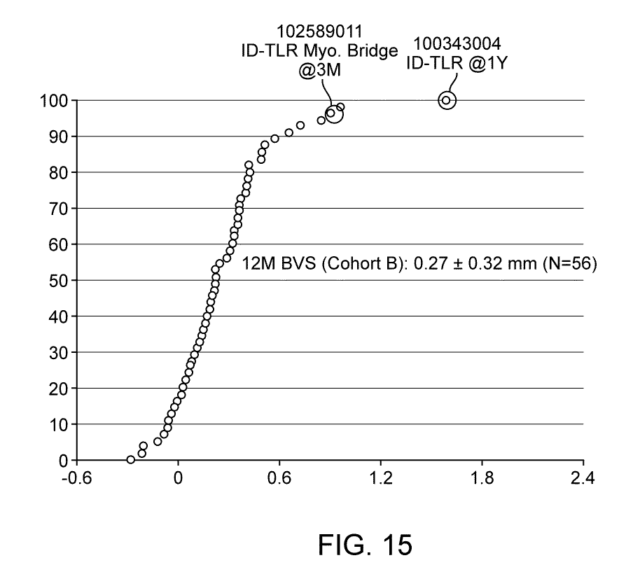

FIG. 15 depicts the evolution of late luminal loss over time for ABSORB Cohort B at 1 year (with events) versus 3 year follow-up (with events) for 56 patients. Late loss is 0.27±0.32 mm (N=56 patients) at 1 year.

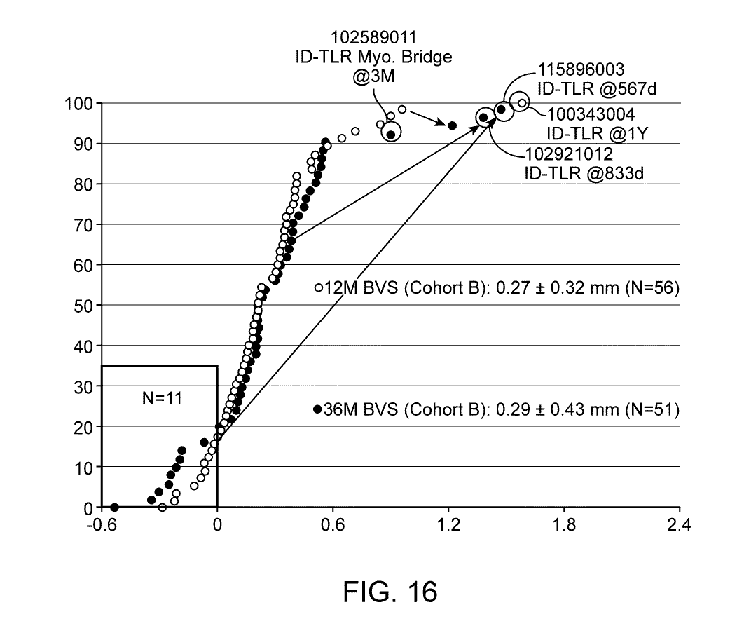

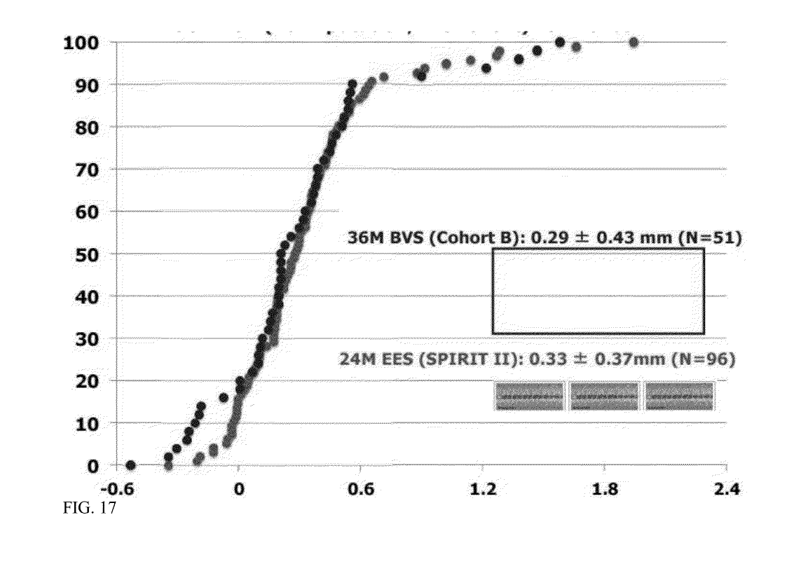

FIG. 16 depicts the evolution of late luminal loss over time for ABSORB at 1 year (lighter color dots, with events) versus ABSORB 3 years (darker color dots, with events). Late loss is 0.29±0.43 mm (N=51 patients) at 3 years.

FIG. 17 depicts the evolution of late luminal loss over time for ABSORB at 3 years follow-up (darker color dots, no imputation, with event) versus Xience V at 2 years follow-up (lighter color dots) everolimus eluting stent (EES) (Spirit II trial) Late loss is 0.33±0.37 mm (N=37 patients) at 2 years.

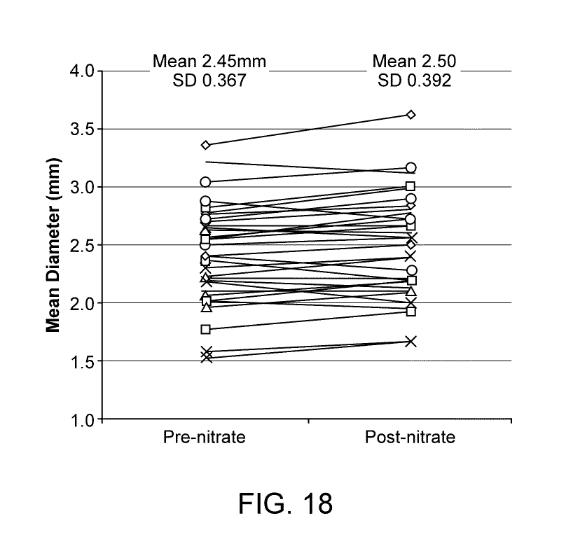

FIG. 18 shows the mean lumen diameter before and after addition of nitrate, a vasodilator, sometime after baseline in the scaffolded segment for 47 patients. FIG. 18 shows dilation of the scaffold segment after baseline, which demonstrates return of vasomotion to the scaffolded segment.

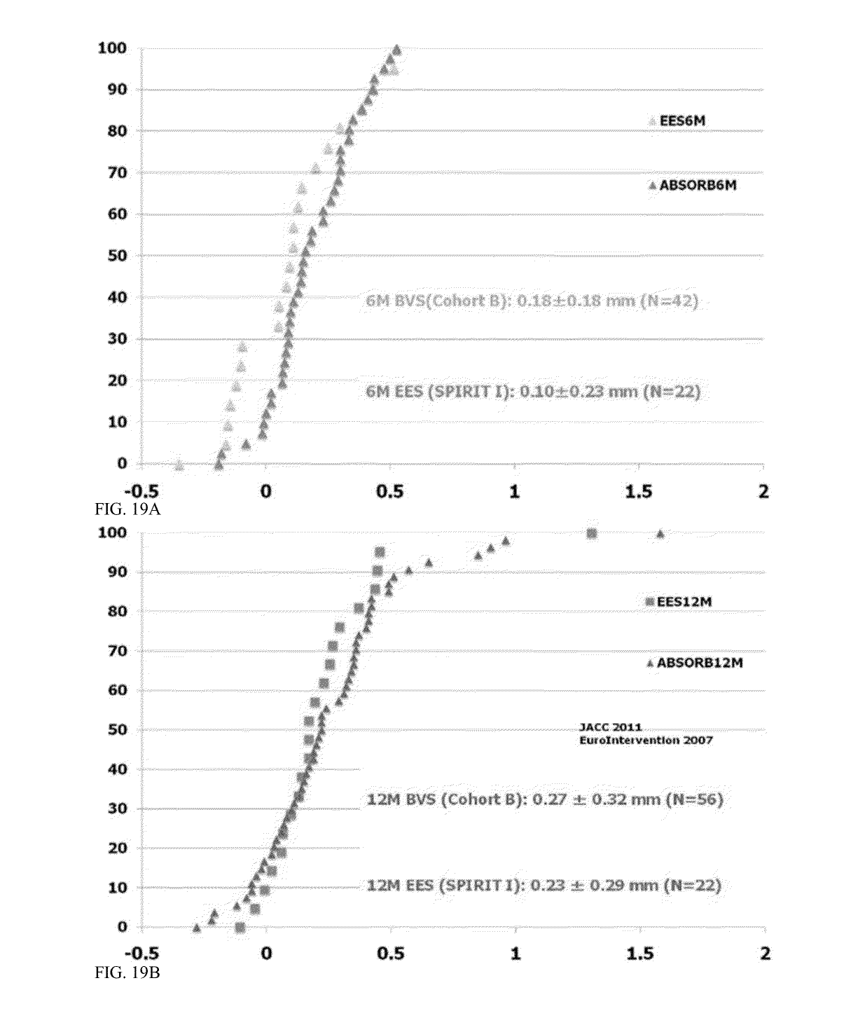

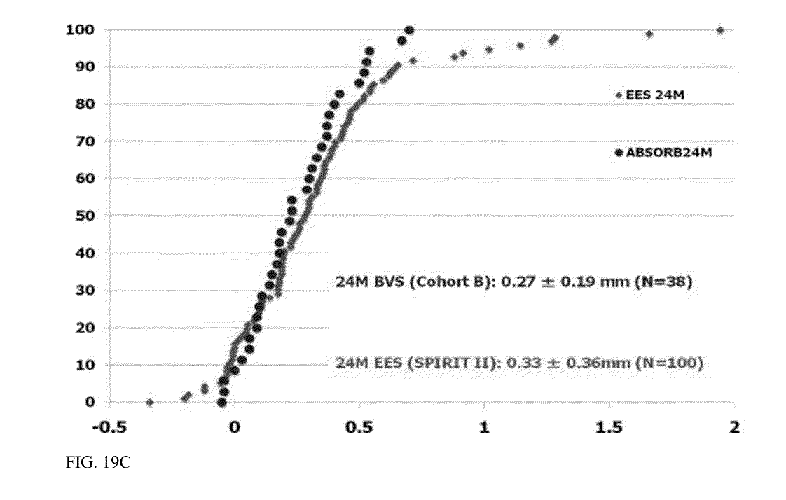

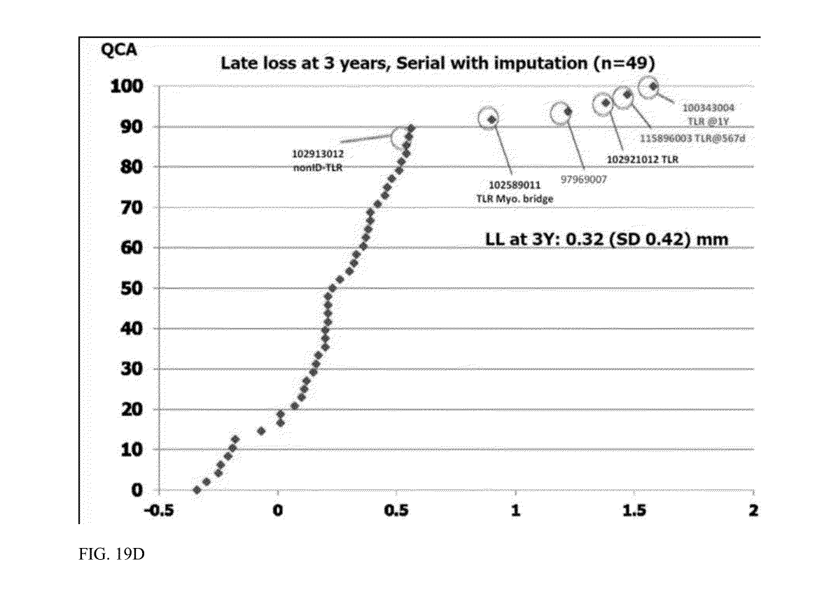

*FIG. 19A-D depicts QCA results showing the evolution of late luminal loss over time for ABSORB at 6 months, 1 year, 2 years, and 3 years follow-up. In FIGS. 19A-C, the ABSORB result is compared to the EES at the same follow-up points. In FIG. 19A, ABSORB is lighter symbol. Late loss is 0.27±0.32 mm (N=56 patients) at 1 year. The corresponding late loss for each is shown in the Figures.

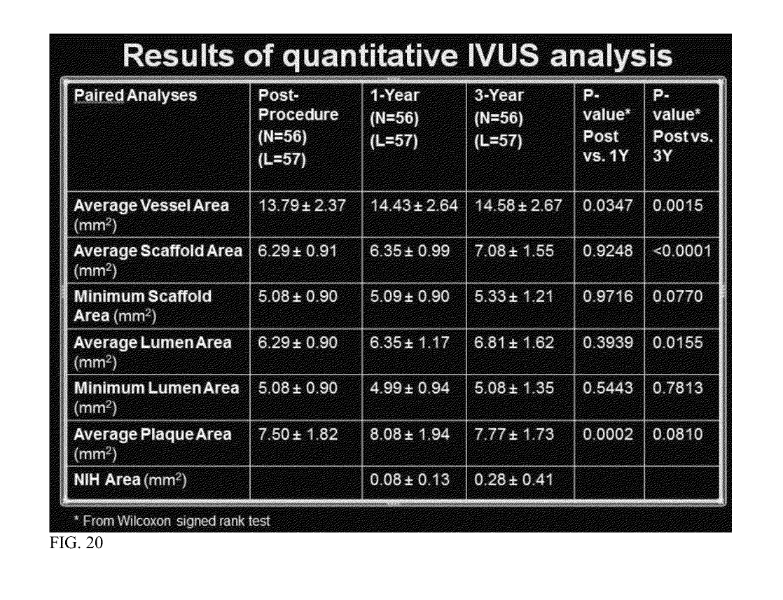

FIG. 20 is table including results of quantitative IVUS analysis of ABSORB for 6 months, 1 year, 2 years, and 3 years follow-up.

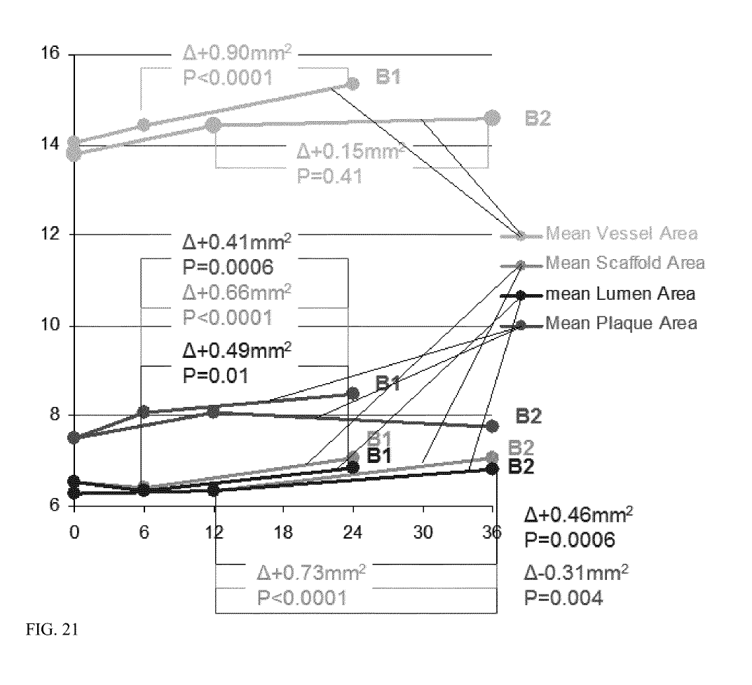

FIG. 21 depicts serial quantitative IVUS analysis for ABSORB of the mean vessel area, mean scaffold area, mean lumen area, and mean plaque area for Group B1 between baseline and 2 years and Group B2 between baseline and 3 years.

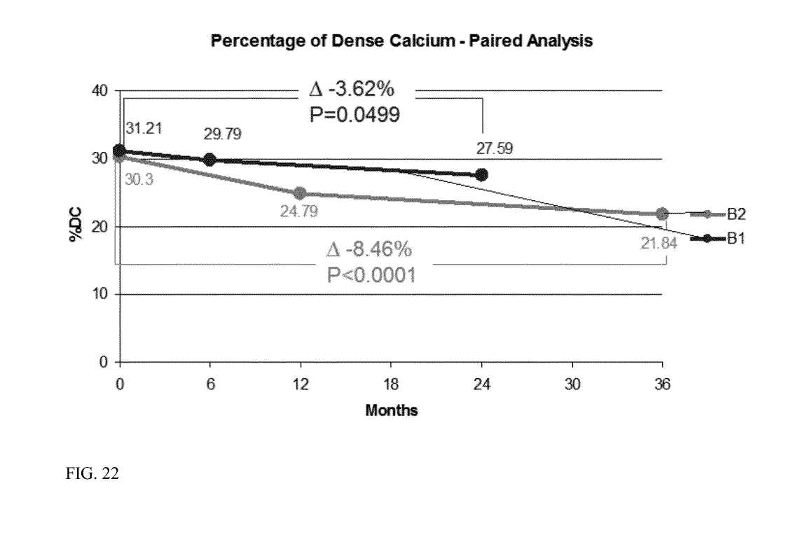

FIG. 22 depicts the results of serial IVUS-VH analysis for percent of dense calcium for Group B1 between baseline and 2 years and Group B2 between baseline and 36 months.

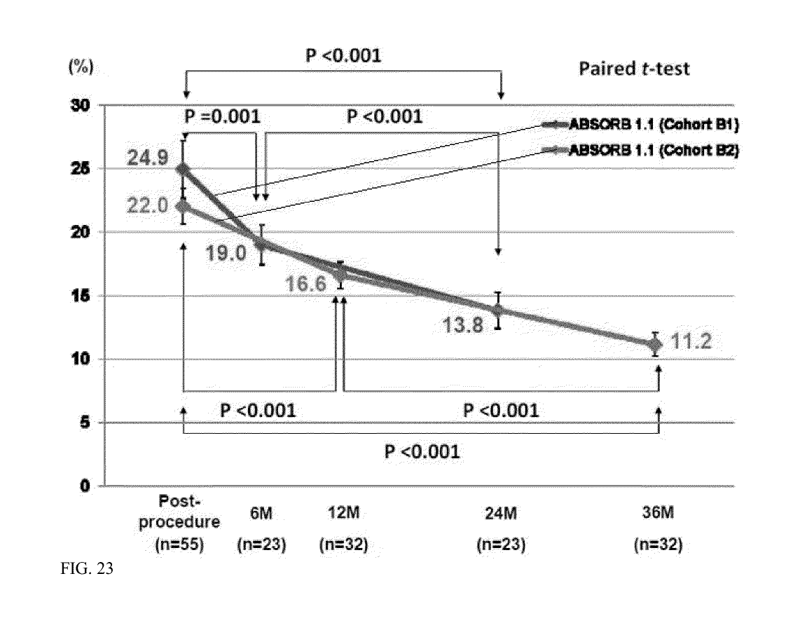

FIG. 23 depicts changes in percentage hyperechogenic area (HEA) for ABSORB 1.1, Cohorts B1, and B2.

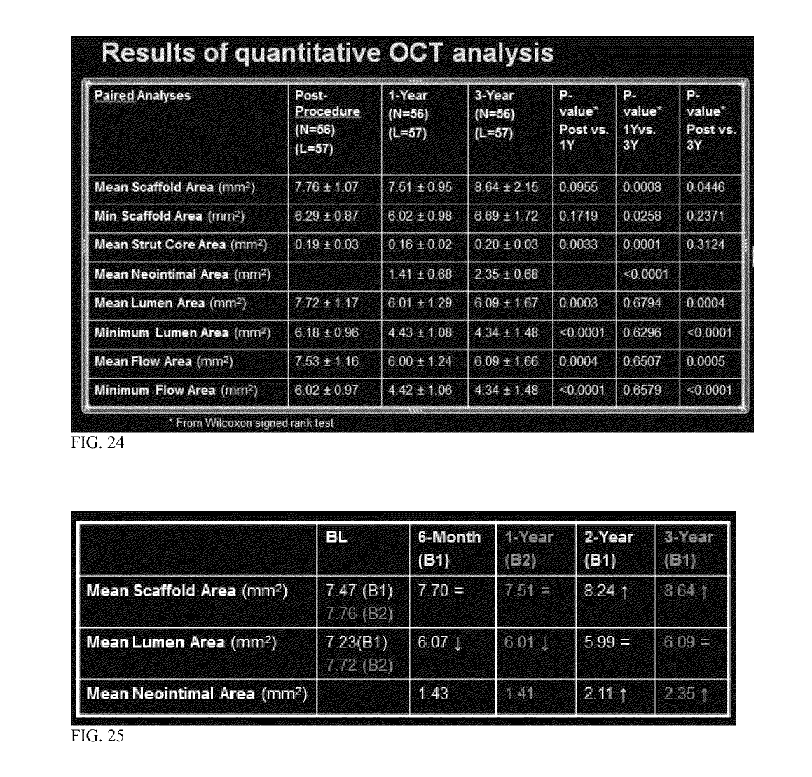

FIG. 24 is a table including results of quantitative OCT analysis for ABSORB post-procedure and for 1 year and 3 years follow-up.

FIG. 25 is a table including results for mean scaffold area, mean lumen area, and mean neointimal area from quantitative OCT analysis for ABSORB at 6 months, 1 year, 2 years, and 3 years follow-up.

The ABSORB EXTEND study is a single-arm trial evaluating Absorb in patients with more complex heart disease. Data from 450 patients enrolled in this trial showed that the rates of MACE at one year were slightly lower than a best-in-class DES. In an analysis of 119 patients with diabetes from the EXTEND trial, rates of MACE were the same in patients with and without diabetes, a promising finding as event rates are typically higher in patients with diabetes when compared to patients without diabetes.

The prevailing mechanism of degradation of many bioabsorbable polymers is chemical hydrolysis of the hydrolytically unstable backbone. In a bulk degrading polymer, the polymer is chemically degraded throughout the entire polymer volume. As the polymer degrades, the molecular weight decreases. The reduction in molecular weight results in changes in mechanical properties (e.g., strength) and stent properties. For example, the strength of the scaffold material and the radial strength of the scaffold are maintained for a period of time followed by a gradual or abrupt decrease. The decrease in radial strength is followed by a loss of mechanical integrity and then erosion or mass loss. Mechanical integrity loss is demonstrated by cracking and by fragmentation. Enzymatic attack and metabolization of the fragments occurs, resulting in a rapid loss of polymer mass.

The behavior of a bioabsorbable stent upon implantation can divided into three stages of behavior. In stage I, the stent provides mechanical support. The radial strength is maintained during this phase. Also during this time, chemical degradation occurs which decreases the molecular weight. In stage II, the scaffold experiences a loss in strength and mechanical integrity. In stage III, significant mass loss occurs after hydrolytic chain scission yields water-soluble low molecular weight species.

The scaffold in the first stage provides the clinical need of providing mechanical support to maintain patency or keep a vessel open at or near the deployment diameter. In some treatments, the patency provided by the scaffold allows the stented segment of the vessel to undergo positive remodeling at the increased deployed diameter. Remodeling refers generally to structural changes in the vessel wall that enhances its load-bearing ability so that the vessel wall in the stented section can maintain an increased diameter in the absence of the stent support. A period of patency is required in order to obtain permanent positive remodeling.

The manufacturing process of a bioabsorbable scaffold includes selection of a bioabsorbable polymer raw material or resin. Detailed discussion of the manufacturing process of a bioabsorbable stent can be found elsewhere, e.g., U.S. Patent Publication No. 20070283552. The fabrication methods of a bioabsorbable stent can include the following steps:

(1) forming a polymeric tube from a biodegradable polymer resin using extrusion,

(2) radially deforming the formed tube to increase radial strength,

(3) forming a stent scaffolding from the deformed tube by laser machining a stent pattern in the deformed tube with laser cutting, in exemplary embodiments, the strut thickness can be 100-200 microns, or more narrowly, 120-180, 130-170, or 140-160 microns,

(4) optionally forming a therapeutic coating over the scaffolding,

(5) crimping the stent over a delivery balloon, and

(6) sterilization with election-beam (E-beam) radiation.

Poly(L-lactide) (PLLA) is attractive as a stent material due to its relatively high strength and rigidity at human body temperature, about 37° C. Since it has a glass transition temperature between about 60 and 65° C. (Medical Plastics and Biomaterials Magazine, March 1998), it remains stiff and rigid at human body temperature. This property facilitates the ability of a PLLA stent scaffold to maintain a lumen at or near a deployed diameter without significant recoil (e.g., less than 10%). In general, the Tg of a semicrystalline polymer can depend on its morphology, and thus how it has been processed. Therefore, Tg refers to the Tg at its relevant state, e.g., Tg of a PLLA resin, extruded tube, expanded tube, and scaffold.

In general, a scaffold can be made of a bioresorbable aliphatic polyester. Additional exemplary biodegradable polymers for use with a bioabsorbable polymer scaffolding include poly(D-lactide) (PDLA), polymandelide (PM), polyglycolide (PGA), poly(L-lactide-co-D,L-lactide) (PLDLA), poly(D,L-lactide) (PDLLA), poly(D,L-lactide-co-glycolide) (PLGA) and poly(L-lactide-co-glycolide) (PLLGA). With respect to PLLGA, the stent scaffolding can be made from PLLGA with a mole % of GA between 5-15 mol %. The PLLGA can have a mole % of (LA:GA) of 85:15 (or a range of 82:18 to 88:12), 95:5 (or a range of 93:7 to 97:3), or commercially available PLLGA products identified as being 85:15 or 95:5 PLLGA. The examples provided above are not the only polymers that may be used. Many other examples can be provided, such as those found in Polymeric Biomaterials, second edition, edited by Severian Dumitriu; chapter 4.

Polymers that are more flexible or that have a lower modulus than those mentioned above may also be used. Exemplary lower modulus bioabsorbable polymers include, polycaprolactone (PCL), poly(trimethylene carbonate) (PTMC), polydioxanone (PDO), poly(4-hydroxy butyrate) (PHB), and poly(butylene succinate) (PBS), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of the blend can include 1-5 wt % of low modulus polymer.

The BVS scaffolds are coated with a polymer mixture that includes Everolimus, an antiproliferative agent. In general, the anti-proliferative agent can be a natural proteineous agent such as a cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin actinomycin X1, and actinomycin C1), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N-1-tetrazolyl)-rapamycin (ABT-578 manufactured by Abbott Laboratories, Abbott Park, Ill.), prodrugs thereof, co-drugs thereof, and combinations thereof.

An anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax ä (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, and genetically engineered epithelial cells. The foregoing substances are listed by way of example and are not meant to be limiting. Other active agents which are currently available or that may be developed in the future are equally applicable. The scaffold can exclude any of the drugs disclosed herein.

“Baseline” refers to a time immediately after deployment of a scaffold to a target diameter in a vessel or at a time after deployment long enough to make measurements on the newly deployed scaffold.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semi-crystalline polymer is exposed to an increasing temperature, the coefficient of expansion and the heat capacity of the polymer both increase as the temperature is raised, indicating increased molecular motion. As the temperature is increased, the heat capacity increases. The increasing heat capacity corresponds to an increase in heat dissipation through movement. Tg of a given polymer can be dependent on the heating rate and can be influenced by the thermal history of the polymer as well as its degree of crystallinity. Furthermore, the chemical structure of the polymer heavily influences the glass transition by affecting mobility.

The Tg can be determined as the approximate midpoint of a temperature range over which the glass transition takes place. [ASTM D883-90]. The most frequently used definition of Tg uses the energy release on heating in differential scanning calorimetry (DSC). As used herein, the Tg refers to a glass transition temperature as measured by differential scanning calorimetry (DSC) at a 20° C./min heating rate.

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to a change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Strength” refers to the maximum stress along an axis which a material will withstand prior to fracture. The ultimate strength is calculated from the maximum load applied during the test divided by the original cross-sectional area.

“Modulus” may be defined as the ratio of a component of stress or force per unit area applied to a material divided by the strain along an axis of applied force that results from the applied force. The modulus typically is the initial slope of a stress—strain curve at low strain in the linear region.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method of treating vascular disease in a patient comprising:
deploying a bioabsorbable polymer scaffold composed of a plurality of struts at a segment of an artery of a patient,
wherein the segment comprises a scaffolded segment between a proximal and a distal end of the scaffold, a proximal segment proximally adjacent to the proximal end of the scaffold, and a distal segment distally adjacent to the distal end of the scaffold,
wherein the proximal segment exhibits constrictive remodeling between baseline and two years after the deployment, wherein the constrictive remodeling comprises a decrease in a cross-sectional area of the proximal segment.

2. The method of claim 1, wherein the constrictive remodeling is present at 6 months after deployment.

3. The method of claim 1, wherein the constrictive remodeling decreases between 6 months and 1 year after deployment.

4. A method of treating vascular disease in a patient comprising:
deploying a bioabsorbable polymer scaffold composed of a plurality of struts at a segment of an artery of a patient,
wherein the segment comprises a scaffolded segment between a proximal and a distal end of the scaffold, a proximal segment proximally adjacent to the proximal end of the scaffold, and a distal segment distally adjacent to the distal end of the scaffold,
wherein a content of fibrotic and fibrofatty (FF) tissue increases at the distal segment between baseline and two years after the deployment.

5. The method of claim 4, wherein the increase is at least 40%.

6. A method of treating vascular disease in a patient comprising:
deploying a bioabsorbable polymer scaffold composed of a plurality of struts at a segment of an artery of a patient,
wherein the segment comprises a scaffolded segment between a proximal and a distal end of the scaffold, a proximal segment proximally adjacent to the proximal end of the scaffold, and a distal segment distally adjacent to the distal end of the scaffold, and
wherein at baseline there is a difference in a compliance of the scaffolded segment between the proximal segment and the distal segment.

7. The method of claim 6, wherein the difference in compliance disappears between 6 and 12 months after deployment.

8. The method of claim 6, wherein the difference in the compliance between the scaffolded segment and the proximal segment is at least 90% and the difference in the compliance between the scaffolded segment and the distal segment is 10 to 40%.

9. A method of treating vascular disease in a patient comprising:
deploying a bioabsorbable polymer scaffold composed of a plurality of struts at a segment of an artery of a patient, the polymer scaffold expanding during deployment which expands the segment to a target diameter,
wherein vasomotion of the segment of the artery reappears after deployment due to the replacement of the polymer by de novo formation of malleable tissue comprising proteoglycan,
wherein two years after deployment the scaffold area or volume has decreased by less than 10%.

10. The method of claim 9, wherein late lumen enlargement occurs after deployment which comprises an increase in the scaffold area or volume two years after deployment which is associated with wall thinning, without expansive remodeling.

11. The method of claim 10, wherein the late enlargement of the scaffold is facilitated by the intraluminal expansive force of the systolic/diastolic lumen wall stress.
